(19) United States
(12) Patent Application Publication
(10) Pub. No.: US 2012/0077734 A1
(43) Pub. Date: Mar. 29, 2012

(54) Methods and assays for treating conditions in which macrophages play a pathogenic role

(76) Inventor: Dianne Cox, New Rochelle, NY (US)

(21) Appl. No.: 13/200,087

(22) Filed: Sep. 16, 2011

Related U.S. Application Data
(60) Provisional application No. 61/387,532, filed on Sep. 29, 2010.

(51) Int. Cl. A61K 38/00; A61P 35/00; A61P 29/00; A61P 19/02; C12Q 1/68; A61P 19/10; A61P 35/04; G01N 33/68; G01N 33/53; A61K 31/7088; A61P 9/10

(52) U.S. Cl. 514/1.9; 514/44 A; 514/1.1; 514/19.3; 514/16.8; 514/16.9; 514/19.5; 514/19.4; 514/19.8; 436/86; 436/501; 435/6.12

(57) ABSTRACT

Methods and assays are disclosed for treating a subject with a disease or condition in which macrophages play a pathogenic role using agents that inhibit or down regulate Wiskott-Aldrich syndrome protein (WASP).

### CROSS-REFERENCE TO RELATED APPLICATION

This application claims the benefit of U.S. Provisional Patent Application No. 61/387,532, filed on Sep. 29, 2010, the content of which is herein incorporated by reference.

### STATEMENT OF GOVERNMENT SUPPORT

This invention was made with government support under grant numbers GM 071828 and CA 100324 awarded by the National Institutes of Health. The government has certain rights in the invention.

### BACKGROUND OF THE INVENTION

Throughout this application various publications are referred to in parenthesis. Full citations for these references may be found at the end of the specification. The disclosures of these publications are hereby incorporated by reference in their entirety into the subject application to more fully describe the art to which the subject invention pertains.

Macrophages (Mφs) are essential components of innate immunity; however, they can also pose disadvantages to the host in certain pathological conditions such as chronic inflammatory diseases or cancer. Tumor-associated macrophages (TAMs), which are present in large numbers in many tumors, appear to play an important role in promoting the progression of solid tumors to an invasive, metastatic phenotype where TAMs play multiple roles in the tumor microenvironment to support metastasis, such as proteolysis of extra-cellular matrix, angiogenesis, enhancing carcinoma cell invasion and their intravasation leading to metastasis (reviewed in (Condeelis and Pollard 2006)). However, the precise role of Mφs in each of these steps is still unknown. The actin cytoskeleton of leukocytes plays a central role in the regulation of cell shape, migration and secretion of growth factor and proteolytic enzymes. Wiskott-Aldrich syndrome protein (WASP) is an activator of the actin nucleator Arp2/3 complex in vitro and expressed exclusively in hematopoietic cells. WASP is mutated in patients with Wiskott-Aldrich syndrome, which is a X-linked genetic disease that causes cellular and humoral immunodeficiency in its complete form (Conley, Notarangelo et al. 1999), and is believed to serve as a key integrator between surface receptors and the cytoskeleton of leukocytes. Mφs from Wiskott-Aldrich syndrome patients demonstrate defects in phagocytosis, podosome mediated matrix degradation and directional migration when placed in a gradient of Mφ chemoattractants, such as colony-stimulating factor-1 (CSF-1) (reviewed in (Thrasher and Burns)). Notably, high circulating levels of CSF-1 in breast cancer patients are positively correlated with poor prognosis (Kacinski, Scata et al. 1991; Scholl, Pallud et al. 1994; Scholl, Lidereau et al. 1996) and CSF-1 deficient mice show lower rates of metastasis (Lin, Nguyen et al. 2001). Since Mφ recruitment to tumors and their motility within tumors are required to exert their pathological functions in the tumor microenvironment, WASP deficiency in Mφs may attenuate tumor invasion and metastasis.

The present invention addresses the need for methods and assays for compounds for treating subjects with diseases and conditions in which macrophages play a pathogenic role.

### SUMMARY OF THE INVENTION

The present invention provides methods for treating a subject with a disease or condition in which macrophages play a pathogenic role comprising administering to the subject an agent that inhibits Wiskott-Aldrich syndrome protein (WASP) or down regulates expression of Wiskott-Aldrich syndrome protein (WASP).

The present invention also provides methods for screening for agents for treating a subject with a disease or condition in which macrophages play a pathogenic role, the methods comprising determining whether or not the agent inhibits Wiskott-Aldrich syndrome protein (WASP) or down regulates expression of Wiskott-Aldrich syndrome protein (WASP), wherein an agent that inhibits Wiskott-Aldrich syndrome protein (WASP) or down regulates expression of Wiskott-Aldrich syndrome protein (WASP) is a candidate for treating a subject with a disease or condition in which macrophages play a pathogenic role.

### BRIEF DESCRIPTION OF THE DRAWINGS

FIG. 1A-1D. WASP is required for macrophage-dependent breast carcinoma cell invasion. (A) Schematic illustration of the 3D invasion assay. (B) X-Z reconstruction of confocal images of 3D invasion assay using celltracker green labeled carcinoma cells (MTLn3) cultured on their own or co-cultured with celltracker red labeled primary bone marrow derived macrophages (BMM) isolated from either wild type (WT) or WASP knockout (WASP−/−) mice. Dotted line indicates the arbitrary threshold for measuring invasion at 20 μm (˜2 cell diameter). Quantification of cell tracker dye labeled (C) macrophage and (D) MTLn3 invasion. Fluorescence from cells was recorded at 5 μm step size. Collagen invasion is plotted as fraction of cells where fluorescence intensities above 20 μm are divided by total fluorescence from the entire stack (n=3 mice per genotype, age and sex matched). Error bars signify SEM. *** p<0.001 compared to WT macrophage co-cultured conditions.

FIG. 2A-2C. WASP is required for the secretion of the carcinoma cell chemoattractant EGF. (A) Quantification of the MTLn3 invasion by MTLn3 cells alone, in response to wildtype BMM conditioned media (WT-CM) or conditioned media from WASP deficient mice (+KO CM) p<** 0.01 ***0.001 compared to wild-type macrophage conditioned media (WT CM). (B) Quantification of the MTLn3 invasion by MTLn3 cells alone, with wildtype BMM conditioned media (WT-CM) alone or with control IgG (Ctrl IgG) or with two different neutralizing anti-EGF antibodies generated in goat or rabbit (nEGF-G or nEGF-R). (C) Quantification of the MTLn3 invasion by MTLn3 cells alone, with WASP deficient BMM conditioned media (+KO CM), or KO CM with exogenously added 25 nM EGF. n=3. Error bars signify SEM.

FIG. 3A-3B. WASP is required for spontaneous lung metastasis (A) Average tumor volume on day of experiment from WT, WASP+/− and WASP−/− mice following MTLn3 injection into their mammary fat pads. (B) Lung metastasis was quantified by directly observing the whole lung under fluorescent microscope and counting blue fluorescent MTLn3 within. Inset of fluorescent images are an example of lung metastasis nodule (>5 cells in same location) and single cell counts. (n=5 for WT mice, 11 for WASP+/− and 9 for WASP−/− mice. Error bars signify SEM. * p<0.05 compared to WT mice. FIG. 4A-4C. WASP is required for spontaneous lung metastasis of human MDA-MD231-4173. (A) Quantification of the MTLn3 invasion by MTLn3 cells alone, in response to wildtype BMM (WT) or WASP deficient mice (WASP−/−) ** p<0.01 compared to WT. (B) Average tumor volume on day of experiment from WASP+/− and WASP−/− mice. (C) Lung metastasis was quantified by directly observing the whole lung under fluorescent microscope and counting blue fluorescent MTLn3 within of nodules (>5 cells in same location) and single cell counts. (n=3 for WASP+/− and WASP−/− mice. Error bars signify SEM. p<* 0.05 **0.01 compared to WASP+/− mice.

FIG. 5. WASP is required for efficient macrophage infiltration of tumor in vivo. Multiphoton intravital stack images were taken from the tumor cortex. Quantification of the total number of macrophages in the field from tumor cortex to 100 μm into the tumor. n=3 independent mice.

FIG. 6A-6B. WASP is required for macrophage dependent carcinoma cell invasion in vivo. (A) Schematic illustration of in vivo invasion assay. (B) Quantification of the number of cells collected into the needle after 4 hours with EGF or without EGF (buffer) from wildtype, WASP+/− or WASP−/− mice bearing MTLn3 tumor. Cell counting was performed single blinded where the counter was not aware of the genotype of the mice. n=4 independent WT and 4 WASp−/− mice used) Error bars signify SEM. ** p<0.01 compared to buffer and EGF needle in WASP−/− mice.

FIG. 7. WASP enhances tumor cell motility in vivo. Multiphoton intravital stack images were collected from the tumor cortex=0 μm, where there is no tumor cell in the field to 100 μm into the tumor for 30 min. Quantification of the total number of carcinoma cells (MTLn3) moving per field. n=3 mice per genotype. * p<0.05.

FIG. 8. WASP phosphorylation regulates the paracrine interaction of macrophages with carcinoma cells. Quantification of the MTLn3 invasion by MTLn3 cells alone or in response to control RAW264.7 macrophage (Ctrl), treated with siRNA against WASP (shWASP), shWASP cells reconstituted with Cdc42 binding deficient (H246D), phopho-mimic (Y291E), phospho-deficient (Y291F) and wildtype WASP (WT). n>3 independent experiments. Error bars signify SEM.

### DETAILED DESCRIPTION OF THE INVENTION

The present invention provides a method for treating a subject with a disease or condition in which macrophages play a pathogenic role comprising administering to the subject an agent that inhibits Wiskott-Aldrich syndrome protein (WASP) or down regulates expression of Wiskott-Aldrich syndrome protein (WASP).

The present invention also provides a method for screening for an agent for treating a subject with a disease or condition in which macrophages play a pathogenic role, comprising determining whether or not the agent inhibits Wiskott-Aldrich syndrome protein (WASP) or down regulates expression of Wiskott-Aldrich syndrome protein (WASP), wherein an agent that inhibits Wiskott-Aldrich syndrome protein (WASP) or down regulates expression of Wiskott-Aldrich syndrome protein (WASP) is a candidate for treating a subject with a disease or condition in which macrophages play a pathogenic role.

The disease or condition can be, for example, cancer, chronic inflammatory disease, arthritis, atherosclerosis or osteoporosis. Where the disease is cancer, inhibiting Wiskott-Aldrich syndrome protein (WASP) or down regulating expression of Wiskott-Aldrich syndrome protein (WASP) may inhibit tumor progression and/or metastasis. For example, the agent may inhibit tumor cell invasiveness, inhibit tumor cell metastasis, and/or inhibit tumor vascularization. The cancer can be, for example, a prostate, pancreas, colon, brain, liver, lung, head or neck cancer, or in particular breast cancer.

The agent can, for example, interfere with WASP phosphorylation or block the interaction between WASP and one or more downstream effectors. Many proteins can bind to WASP and regulate its activity such as Cdc42, PIP2, Toca-1, Src family kinases, vasp, CK2, NCK, GRB, CRKL, syndapin, and PSTPIP1. The best characterized downstream effector of WASP is Arp2/3. WASP binding of the Arp2/3 complex activates Arp2/3 actin polymerization activity. WASP also binds to a number of proteins that may regulate additional activities such as vesicle trafficking.

WASP can be inhibited using, for example, a small molecule inhibitor, small interfering RNA, small interfering RNA using shorthairpin RNA, or cell penetrating phosphopeptide or peptide to block the binding of a WASP effector to WASP. It may also be possible to inhibit WASP by inhibiting an upstream activator such as Src family kinases or Cdc42, but blocking these proteins would not only have effects on WASP activity and therefore would be less specific.

WASP is one protein in a family called the WASP/WAVE proteins. WASP is most closely related to the ubiquitously expressed family member N-WASP and then the related WAVE family including WAVE 1, 2 and 3. In one embodiment, human WASP protein has the following amino sequence (NCBI Reference Sequence: NP_{—}000368.1) (Derry et al. 1994) (SEQ ID NO:1):

```
    1 msggpmggrp ggrgapavqq nipstllqdh enqrlfemlg rkcltlatav vqlylalppg
  61 aehwtkehcg avcfvkdnpq ksyfirlygl qagrllweqe lysqlvystp tpffhtfagd
121 dcqaglnfad edeaqafral vqekiqkrnq rqsgdrrqlp ppptpaneer rgglpplplh
181 pggdqggppv gplslglatv diqnpditss ryrglpapgp spadkkrsgk kkiskadiga
241 psgfkhvshv gwdpqngfdv nnldpdlrsl fsragiseaq ltdaetskli ydfiedqggl
301 eavrqemrrq eplpppppps rggnqlprpp ivggnkgrsg plppvplgia pppptprgpp
361 ppgrggpppp pppatgrsgp lpppppgagg ppmppppppp ppppssgngp appplppalv
421 pagglapggg rgalldqirq giqlnktpga pessalqppp qsseglvgal mhvmqkrsra
481 ihssdegedq agdededdew dd
```

In one embodiment, human N-WASP protein has the following amino sequence (NCBI Accession No. BAA20128) (Fukuoka et al. 1997) (SEQ ID NO:2):

```
    1 mssvqqqppp prrvtnvgsl lltpqenesl ftflgkkcvt mssavvqlya adrncmwskk
  61 csgvaclvkd npqrshflri fdikdgkllw eqelynnfvy nsprgyfhtf agdtcqvaln
121 faneeeakkf rkavtdllgr rqrksekrrd ppngpnlpma tvdiknpeit tnrfygpqvn
181 nishtkekkk gkakkkrltk gdigtpsnfq highvgwdpn tgsdlnnldp elknlfdmcg
241 ileaqlkere tlkviydfie ktggveavkn elrrqapppp ppsrggpppp pppphssgpp
301 pppargrgap ppppsrapta appppppsrp svevpppppn rmypppppal pssapsgppp
361 pppsvlgvgp vapppppppp pppgpppppg lpsdgdhqvp ttagnkaall dqiregaqlk
421 kveqnsrpvs csgrdalldq irqgiqlksv adgqestppt paptsgivga lmevmqkrsk
481 aihssdeded eddeedfedd dewed
```

The agent that inhibits or down regulates WASP can be administered to the subject in a pharmaceutical composition comprising a pharmaceutically acceptable carrier. Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. The pharmaceutically acceptable carrier used can depend on the route of administration. The pharmaceutical composition can be formulated for administration by any method known in the art, including but not limited to, oral administration, parenteral administration, intravenous administration, transdermal administration, intranasal administration, and administration through an osmotic mini-pump. The compounds can be applied to the skin, for example, in compositions formulated as skin creams, or as sustained release formulations or patches.

Agents can be tested as possible inhibitors of WASP using, for example, an in vitro actin polymerization assay, which uses purified WASP to stimulate Arp2/3 activity, or a WASP biosensor that uses intramolecular fluorescence resonance energy transfer to report WASP activation in vivo (Cammer et al. 2009). An inhibitor of N-WASP, called wiskostatin, has been developed (Peterson et al., 2004) that also inhibits WASP. However, this inhibitor lowers the intracellular levels of ATP and therefore has non-specific effects. Preferably, an inhibitor will specifically target WASP and not have an effect on N-WASP activity. Compounds that inhibit both WASP and N-WASP can be excluded, for example, by comparing the inhibitory properties on WASP and N-WASP stimulated actin polymerization in vitro or by comparing inhibition of activity using the available WASP and N-WASP biosensors (Lorenz et al., 2004). Western blotting with a WASP specific antibody can be used, for example, to determine expression level, or qRT-PCR can be used to examine message level. Blocking downstream interactions can be examined by co-immunoprecipitation experiments.

The invention also provides an agent identified by any of the methods disclosed herein. The agent can be used, for example, to treat cancer, chronic inflammatory disease, arthritis, atherosclerosis or osteoporosis.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

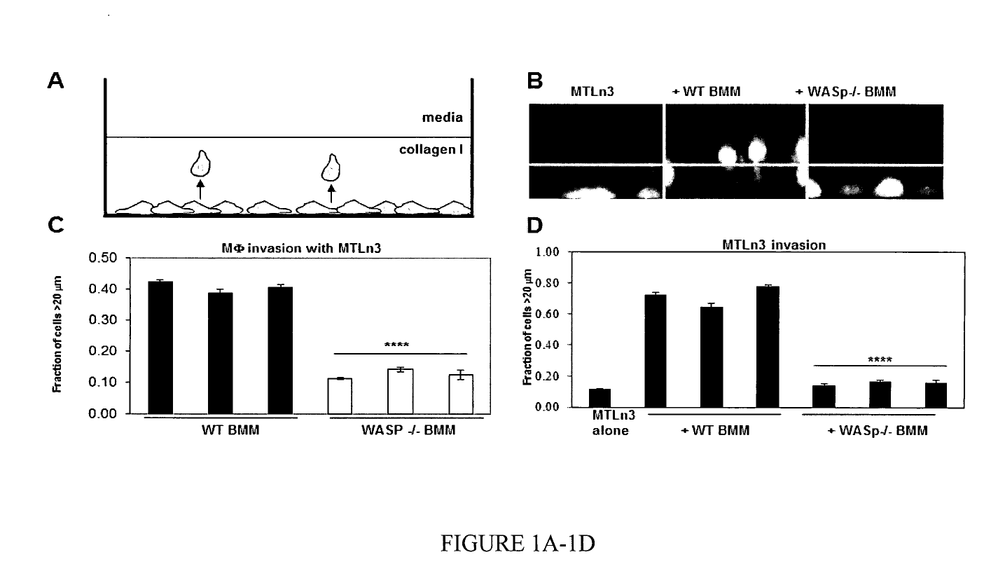

Recent rodent studies have demonstrated the existence of a paracrine loop interaction between epidermal growth factor (EGF) secreting TAMs and CSF-1 secreting tumor cells, which promote mutual migration of both cell types leading to tumor cell invasion and metastasis (Wyckoff, Wang et al. 2004; Goswami, Sahai et al. 2005). To test whether WASP can promote carcinoma cell invasion in 3D, collagen gel matrix was layered over co-cultured WASP-deficient bone marrow derived Mφs (BMM) and highly metastatic breast tumor cells (MTLn3) (FIG. 1). Akin to results from previously published 3D invasion study using Mφ cell lines (Goswami, Sahai et al. 2005), control bone marrow-derived Mφ (BMM) significantly invaded the collagen gel, while the WASP-deficient BMM did not promote MTLn3 invasion at all (FIG. 1). Consistent with the paracrine interaction between Mφ and tumor cells, MTLn3 invasion was significantly enhanced by the presence of control BMM, while the WASP-deficient BMM did not promote MTLn3 invasion (FIG. 1D).

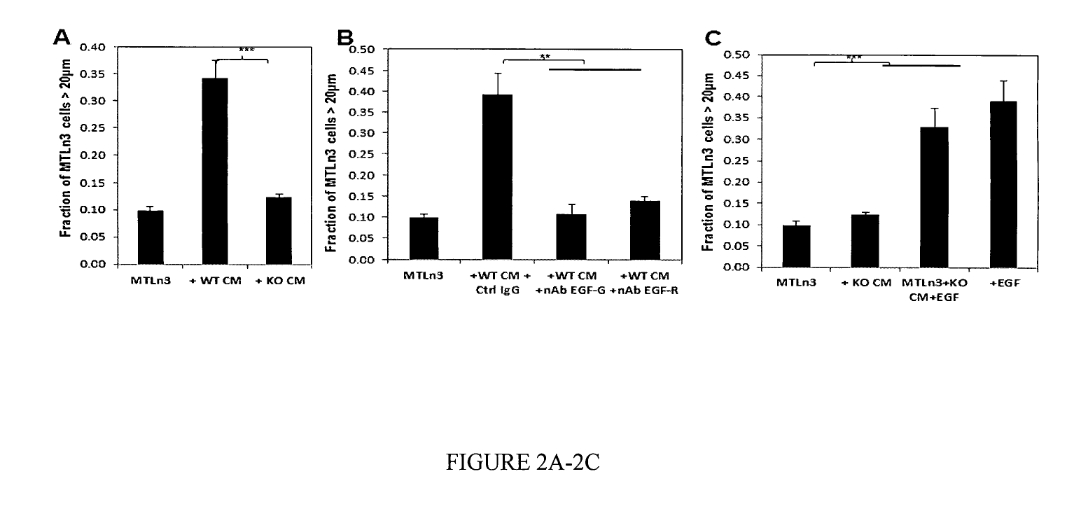

Interestingly, the defect in MTLn3 invasion could be partially rescued by the addition of conditioned media from control BMM but not with conditioned media from WASP-deficient BMM (FIG. 2A). Since neutralizing antibodies against EGF attenuated the effect of conditioned media and addition of EGF to WASP deficient conditioned media restored carcinoma cell invasion, this indicated that WASP also plays a role in secretion of carcinoma cell chemoattractants (FIGS. 2B and 2C). To confirm that the effect of co-culture was in fact due to the proposed paracrine interaction, the effect of adding small molecule inhibitors of CSF-1R and EGFR were tested. The addition of either inhibitor effectively blocked the effect of co-culture (data not shown). These findings support the hypothesis that both WASP dependent secretion and macrophage migration are important in supporting the high level of carcinoma invasiveness.

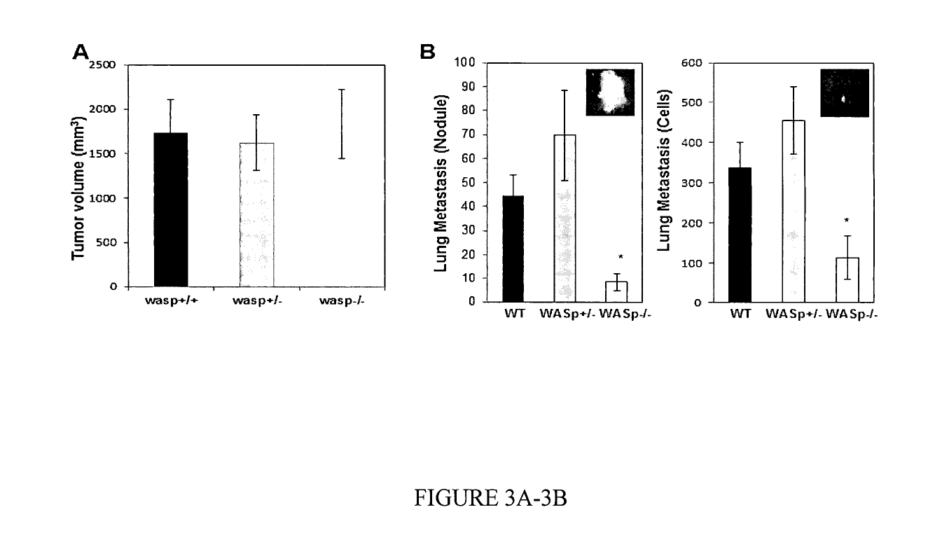

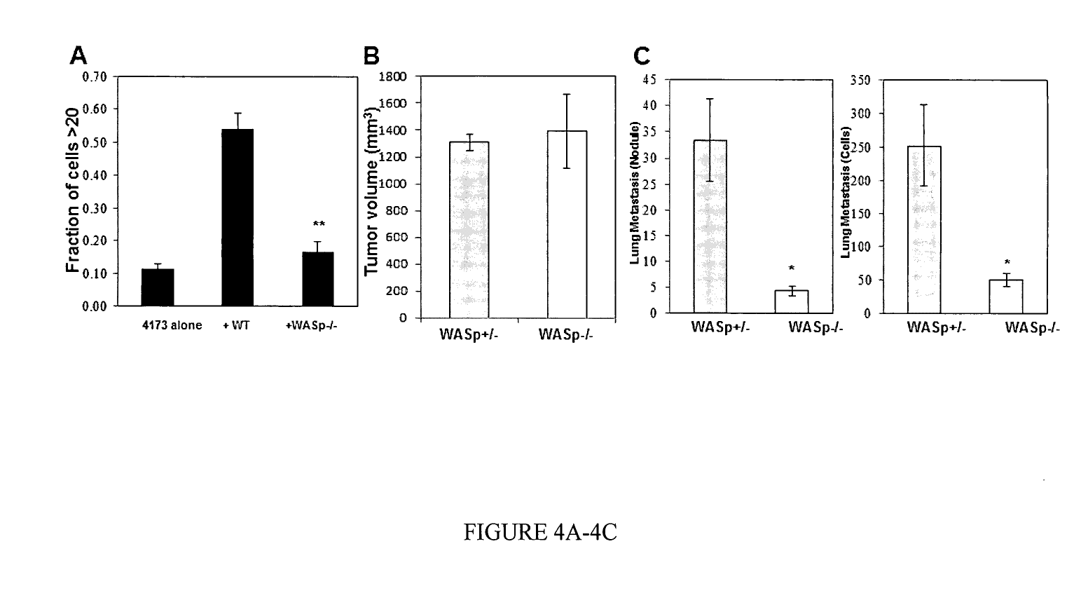

Taken together, these findings support the hypothesis that WASP plays a role in the paracrine interaction between Mφs and carcinoma cells, leading to the co-migration of both cell types required to induce the high level of carcinoma cell invasiveness observed in vitro and in vivo. Therefore, xenograft competent (Rag2−/−)/WASP-deficient mice were generated with GFP expressing Mφs (under the control of the cfms promotor), and MTLn3-CFP was injected into the mammary fat pad. Primary tumor growth rate was not significantly different between control and WASP-deficient mice (FIG. 3A). However, lungs from WASP-deficient mice contained significantly fewer metastatic cells compared to control mice, as assessed by CFP fluorescence (FIG. 3B). These results were confirmed using the highly invasive human metastatic breast cancer cell line, MDA-MB-231-4173 (Lu and Kang 2009). Correspondingly, WASP-deficient macrophages failed to promote MDA-MB-231-4173 invasion in vitro and metastasis was significantly reduced in WASp-deficient mice injected with MDA-MB-231 cells, indicating WASp plays a key role in the paracrine interaction that leads to tumor progression and metastasis using a human model system (FIG. 4).

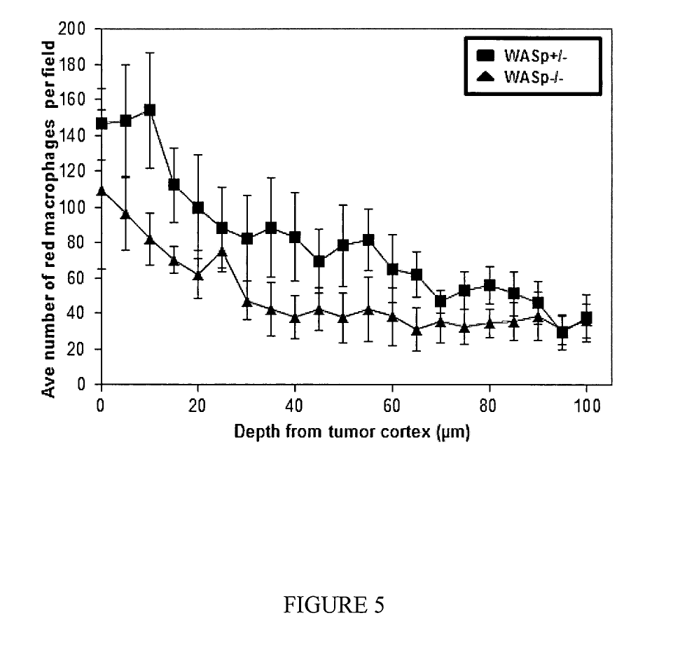

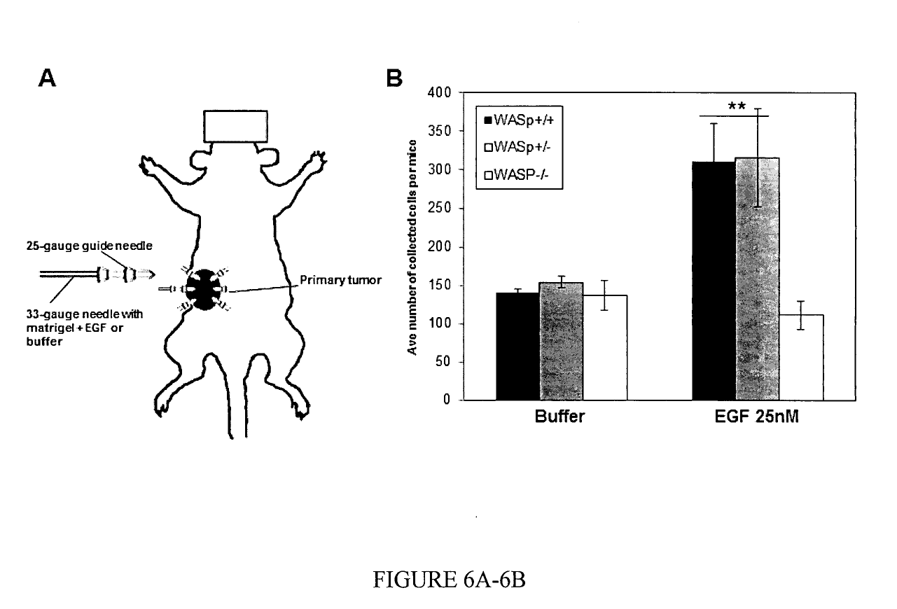

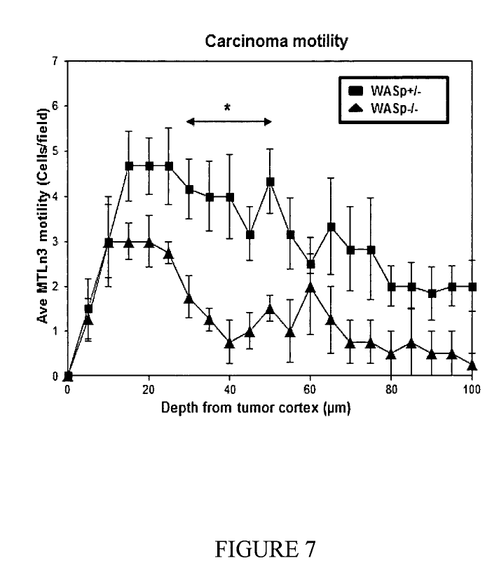

The presence and absence of WASP in tumors was further characterized to determine the precise defect in the absence of WASP that led to reduced metastasis. Both histological samples and intravital imaging of the fluorescent carcinoma cells and macrophages showed a small but significant reduction in the number of TAMs in tumors of WASP-deficient mice compared to control mice (FIG. 5). Carcinoma cell invasiveness in the tumor was monitored and analyzed using a well established in vivo needle invasion assay that mimics the invasion of carcinoma cells into a blood vessel, according to Wyckoff et al. (Wyckoff, Segall et al. 2000). Consistent with the block in the paracrine interaction observed in vitro in the absence of WASP, EGF significantly enhanced the number of carcinoma cells collected into a needle from tumor bearing control mice while there was no difference in the number of cells collected into needle containing buffer alone or EGF from tumor bearing WASP-deficient mice (FIG. 6). Additionally, a reduction was also observed in the motility of carcinoma cells in the tumors of WASP deficient mice as compared with control mice (FIG. 7). These data are consistent with the in vitro data and support a role for WASP in regulating the ability of TAMs to enhance carcinoma cell motilty and invasion leading to metastatic spread.

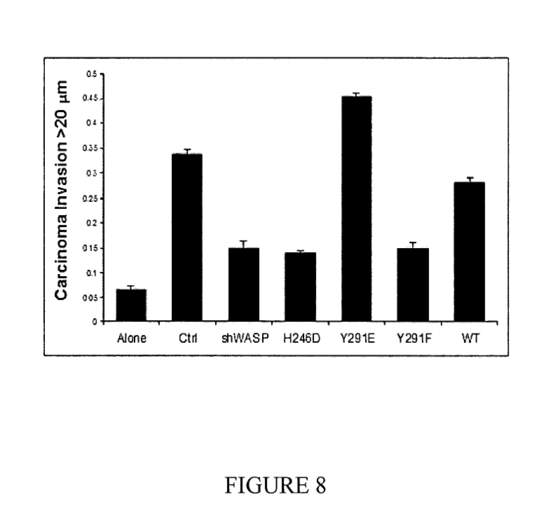

TAMs have also been proposed to aid tumor progression by breakdown of the extracellular matrix and by promoting angiogenesis. WASP is required for the formation of podosomes, F-actin rich ventral adhesion structures of leukocytes, including Mφs dendritic cells and osteoclasts that mediate matrix degradation (reviewed in (Linder and Aepfelbacher 2003)). Consistent with a role for WASP in matrix degradation, resting or activated macrophages from WASP-deficient mice show reduced ability to degrade matrix (Isaac, Ishihara et al.). Furthermore, using a Mφ cell line in which levels of endogenous WASP has been reduced using siRNA and then reconstituted to express mutant forms of WASP, it was recently shown that phosphorylation of WASP regulates many of WASP functions such as phagocytosis, matrix degradation and chemotaxis (Dovas, Gevrey et al. 2009; Park and Cox 2009). Consistent with key role of WASP phosphorylation in Mφ function, cells that express either phospho-deficient or phospho-mimicking forms of WASP demonstrate altered carcinoma cell invasion in vitro (FIG. 8). These results suggests that regents that interfere with WASP phosphorylation or would block the interaction between WASP and downstream effectors might also be effective in inhibiting the paracrine interaction between TAMs and carcinoma cells in vivo. In addition, reduced tumor vascularization was observed in the tumors from WASP-deficient mice suggesting a role for WASP in tumor angiogenesis. CSF-1 dependent TAMs are known to promote tumor angiogeneis through the secretion of VEFG, enhancing tumor progression (Lin, Li et al. 2007; Ojalvo, King et al. 2009).

The present data demonstrate that WASP plays a major role in macrophage-mediated tumor progression and metastasis suggesting WASP inhibition may have therapeutic benefit in the treatment of breast tumor progression.

Breast cancer continues to be the most frequently diagnosed and second leading cause of death from cancer in women. While early detection and advent of combination therapy show an increase in mortality, the presence of metastasis generally implies poor prognosis. While chemotherapy continues to get more sophisticated and specific, frequently tumor cells develop resistance to these drugs resulting in recurrence and metastasis. Therefore, targeting an important molecule in cancer progression that is expressed in more stable, non-cancerous cells host cells is a promising addition and/or an alternative to the current therapeutic regimen since the host cells are much less likely to develop resistance. WASP may be a viable candidate since WASP is an important molecule in tumor progression and yet it is expressed exclusively in hematopoietic cells. In addition, targeted therapeutics against WASP may also be applicable to chronic inflammatory diseases where Mφs appear to play pathogenic roles such as arthritis and atherosclerosis. In addition, since this protein is important in mediating osteoclast function, this invention may also be applicable to the treatment of osteoporosis.

### REFERENCES

Cammer M, Gevrey J C, Lorenz M, Dovas A, Condeelis J, Cox D. The mechanism of CSF-1-induced Wiskott-Aldrich syndrome protein activation in vivo: a role for phosphatidylinositol 3-kinase and Cdc42. J Biol Chem. 2009 Aug. 28; 284(35):23302-11. Epub 2009 Jun. 26.
Condeelis, J. and J. W. Pollard (2006). Macrophages: obligate partners for tumor cell migration, invasion, and metastasis. Cell 124(2): 263-6.
Conley, M. E., L. D. Notarangelo, et al. (1999). Diagnostic criteria for primary immunodeficiencies. Representing PAGID (Pan-American Group for Immunodeficiency) and ESID (European Society for Immunodeficiencies). Clin Immunol 93(3): 190-7.
Derry, J. M., Ochs, H. D. and Francke, U. Isolation of a novel gene mutated in Wiskott-Aldrich syndrome. Cell 78 (4): 635-644 (1994).
Dovas, A., J. C. Gevrey, et al. (2009). Regulation of podosome dynamics by WASp phosphorylation: implication in matrix degradation and chemotaxis in macrophages. J Cell Sci 122(Pt 21): 3873-82.
Fukuoka, M., Miki, H. and Takenawa, T. Identification of N-WASP homologs in human and rat brain. Gene 196 (1-2): 43-48 (1997).
Goswami, S., E. Sahai, et al. (2005). Macrophages promote the invasion of breast carcinoma cells via a colony-stimulating factor-1/epidermal growth factor paracrine loop. Cancer Res 65(12): 5278-83.
Isaac, B. M., D. Ishihara, et al. N-WASP has the ability to compensate for the loss of WASP in macrophage podosome formation and chemotaxis. Exp Cell Res. 2010 Dec. 10; 316(20):3406-16. Epub 2010 Jun. 27.
Kacinski, B. M., K. A. Scata, et al. (1991). FMS (CSF-1 receptor) and CSF-1 transcripts and protein are expressed by human breast carcinomas in vivo and in vitro. Oncogene 6(6): 941-52.
Lorenz M, Yamaguchi H, Wang Y, Singer R H, Condeelis J (2004). “Imaging sites of N-WASP activity in lamellipodia and invadopodia of carcinoma cells.” Curr Biol. 14(8):697-703.
Lin, E. Y., J. F. Li, et al. (2007). “Vascular endothelial growth factor restores delayed tumor progression in tumors depleted of macrophages.” Mol Oncol 1(3): 288-302.
Lin, E. Y., A. V. Nguyen, et al. (2001). “Colony-stimulating factor 1 promotes progression of mammary tumors to malignancy.” J Exp Med 193(6): 727-40.
Linder, S. and M. Aepfelbacher (2003). “Podosomes: adhesion hot-spots of invasive cells.” Trends Cell Biol 13(7): 376-85.
Lu, X. and Y. Kang (2009). “Efficient acquisition of dual metastasis organotropism to bone and lung through stable spontaneous fusion between MDA-MB-231 variants.” Proc Natl Acad Sci USA 106(23): 9385-90.
Ojalvo, L. S., W. King, et al. (2009). “High-density gene expression analysis of tumor-associated macrophages from mouse mammary tumors.” Am J Pathol 174(3): 1048-64.
Park, H. and D. Cox (2009). “Cdc42 regulates Fc gamma receptor-mediated phagocytosis through the activation and phosphorylation of Wiskott-Aldrich syndrome protein (WASP) and neural-WASP.” Mol Biol Cell 20(21): 4500-8.
Peterson J R, Bickford L C, Morgan D, Kim A S, Ouerfelli O, Kirschner M W, Rosen M K (2004). “Chemical inhibition of N-WASP by stabilization of a native autoinhibited conformation.” Nat Struct Mol Biol. 11(8):747-55.
Scholl, S. M., R. Lidereau, et al. (1996). “Circulating levels of the macrophage colony stimulating factor CSF-1 in primary and metastatic breast cancer patients. A pilot study.” Breast Cancer Res Treat 39(3): 275-83.
Scholl, S. M., C. Pallud, et al. (1994). “Anti-colony-stimulating factor-1 antibody staining in primary breast adenocarcinomas correlates with marked inflammatory cell infiltrates and prognosis.” J Natl Cancer Inst 86(2): 120-6.
Thrasher, A. J. and S. O. Burns “WASP: a key immunological multitasker.” Nat Rev Immunol 10(3): 182-92.
Wyckoff, J., W. Wang, et al. (2004). “A paracrine loop between tumor cells and macrophages is required for tumor cell migration in mammary tumors.” Cancer Res 64(19): 7022-9.
Wyckoff, J. B., J. E. Segall, et al. (2000). “The collection of the motile population of cells from a living tumor.” Cancer Res 60(19): 5401-4.

What is claimed is:

1. A method for treating a subject with a disease or condition in which macrophages play a pathogenic role comprising administering to the subject an agent that inhibits Wiskott-Aldrich syndrome protein (WASP) or down regulates expression of Wiskott-Aldrich syndrome protein (WASP).

2. The method of claim 1, wherein the disease or condition is cancer, chronic inflammatory disease, arthritis, atherosclerosis or osteoporosis.

3. The method of claim 1, wherein the disease is prostate, pancreas, colon, brain, liver, lung, head, neck or breast cancer.

4. The method of claim 1, wherein the disease is cancer and wherein the agent inhibits tumor cell invasiveness, inhibits tumor cell metastasis, and/or inhibits tumor vascularization.

5. The method of claim 1, wherein the agent interferes with WASP phosphorylation or blocks an interaction between WASP and a downstream effector.

6. The method of claim 1, wherein the agent does not inhibit or down regulate N-WASP.

7. A method for screening for an agent for treating a subject with a disease or condition in which macrophages play a pathogenic role, comprising determining whether or not the agent inhibits Wiskott-Aldrich syndrome protein (WASP) or down regulates expression of Wiskott-Aldrich syndrome protein (WASP), wherein an agent that inhibits Wiskott-Aldrich syndrome protein (WASP) or down regulates expression of Wiskott-Aldrich syndrome protein (WASP) is a candidate for treating a subject with a disease or condition in which macrophages play a pathogenic role.

8. The method of claim 7, comprising determining whether or not the agent inhibits or down regulates N-WASP, wherein an agent that inhibits or down regulates WASP but does not inhibit or down regulate N-WASP is a candidate for treating a subject with a disease or condition in which macrophages play a pathogenic role.

9. The method of claim 7, wherein the disease or condition is cancer, chronic inflammatory disease, arthritis, atherosclerosis or osteoporosis.

10. The method of claim 7, wherein the disease is prostate, pancreas, colon, brain, liver, lung, head, neck or breast cancer.

11. The method of claim 7, wherein the disease is cancer and wherein the agent inhibits tumor cell invasiveness, inhibits tumor cell metastasis, and/or inhibits tumor vascularization.

12. The method of claim 7, wherein the agent interferes with WASP phosphorylation or blocks an interaction between WASP and a downstream effector.


SEQUENCE LISTING (2 sequences)

SEQ ID NO: 1 - protein, 502 aa, Homo sapiens
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 2 - protein, 505 aa, Homo sapiens
MSSVQQQPPPPRRVTNVGSLLLTPQENESLFTFLGKKCVTMSSAVVQLYAADRNCMWSKKCSGVACLVKDNPQRSHFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKGDIGTPSNFQHIGHVGWDPNTGSDLNNLDPELKNLFDMCGILEAQLKERETLKVIYDFIEKTGGVEAVKNELRRQAPPPPPPSRGGPPPPPPPPHSSGPPPPPARGRGAPPPPPSRAPTAAPPPPPPSRPSVEVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED